(19) United States
(12) Patent Application Publication
(10) Pub. No.: US 2020/0179220 A1
(43) Pub. Date: Jun. 11, 2020

(54) CUPPING DEVICE AND METHOD FOR FACIAL AND SKIN TREATMENT

(71) Applicant: JJJ Health LLC, New York, NY (US)

(72) Inventor: Jennifer Jablow, New York, NY (US)

(21) Appl. No.: 16/707,792

(22) Filed: Dec. 9, 2019

Related U.S. Application Data
(60) Provisional application No. 62/777,290, filed on Dec. 10, 2018.

(51) Int. Cl. A61H 9/00

(52) U.S. Cl. CPC A61H 9/0057; A61H 2201/10; A61H 2205/022; A61N 5/0616; A61H 2201/0153; A61H 2201/1685; A61H 2201/02

(57) ABSTRACT

A vacuum suction handheld device with a cup attachment at the end that contacts the skin with negative pressure and light and gently glides across the treatment area. The goal of this is to relieve muscle tension, encourage lymphatic drainage, and soften fine lines/wrinkles by opening up blood vessels under the skin creating a plumping effect. The device can also have red or blue light technology, vibration and hot/cold added to it.

### CROSS-REFERENCE TO RELATED APPLICATIONS

This application claims priority to and the benefit of U.S. Provisional Patent Application Ser. No. 62/777,290 filed Dec. 10, 2018, the disclosure of which is incorporated herein by reference in its entirety.

### FIELD

The aspects of the disclosed embodiments generally relate to skin care devices and methods related thereto.

### BACKGROUND

The ancient art of cupping has been used in Chinese medicine for hundreds of years. The process can gently, manually suck up sections of the skin, for example, the face as well as improve skin circulation, encourage lymph drainage, tones tissue that is flaccid, and relaxes tightness. It can also increase the diffusion and receptivity of skin cell nutrients, stimulates collagen and elastin and contribute to the process of restoring new skin tissue.

Cupping may aid with the relief of pain and inflammation, improve blood flow, contribute to a feeling of relaxation and well-being, as well as treat deep scar tissues in the muscles and connective tissue such as muscle knots and swelling.

A 2014 report in the Journal of Traditional Chinese Medical Sciences entitled “Cupping therapy for acute and chronic pain management: a systematic review of randomized clinical trials” (volume 1, issue 1, Jul. 2014, pp 49-61) found that there was a positive short-term effect of cupping therapy on reducing pain intensity compared with no treatment, heat therapy, or conventional drugs. A 2016 study in the Journal Evidence Based Complement Alternative Medicine entitled “The Effectiveness of Cupping Therapy on Relieving Chronic Neck and Shoulder Pain: A Randomized Controlled Trial” (published online 2016 Mar. 17) also found that cupping was effective at relieving chronic neck and shoulder pain.

Traditionally, the cup can be heated up and placed statically on an area of the skin statically. However, this technique can leave unsightly marks on the cupped area. It would be advantageous to have a skin care treatment that is more efficient and does not leave such unsightly marks on the treated area.

### DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1A is a top front perspective view of a device embodiment of the present disclosure;

FIG. 1B is a front perspective view of the device embodiment of FIG. 1A with the cup attachment of FIGS. 2A-2E;

FIG. 1C is s side view of the device embodiment of FIG. 1A;

FIG. 1D is a bottom front perspective view of the device embodiment of FIG. 1A with the cup attachment of FIGS. 2A-2E;

FIG. 1E is a front view of the device embodiment of FIG. 1A with the cup attachment of FIGS. 2A-2E

FIG. 1F is a back view of the device embodiment of FIG. 1A with the cup attachment of FIGS. 2A-2E;

FIG. 1G is a side view of the device embodiment of FIG. 1A with the cup attachment of FIGS. 2A-2E where the other side is a mirror image;

FIG. 1H is a top view of the device embodiment of FIG. 1A with the cup attachment of FIGS. 2A-2E;

FIG. 1I is a bottom view of the device embodiment of FIG. 1A with the cup attachment of FIGS. 2A-2E;

FIG. 1J is a top front perspective view of the device embodiment of FIG. 1A with the cup attachment of FIGS. 3A-3E;

FIG. 1K is a bottom front perspective view of the device embodiment of FIG. 1A with the cup attachment of FIGS. 3A-3E;

FIG. 1L is a front view of the device embodiment of FIG. 1A with the cup attachment of FIGS. 3A-3E;

FIG. 1M is a back view of the device embodiment of FIG. 1A with the cup attachment of FIGS. 3A-3E;

FIG. 1N is a side view of the device embodiment of FIG. 1A with the cup attachment of FIGS. 3A-3E where the other side is a mirror image;

FIG. 1O is a top view of the device embodiment of FIG. 1A with the cup attachment of FIGS. 3A-3E;

FIG. 1P is a bottom view of the device embodiment of FIG. 1A with the cup attachment of FIGS. 3A-3E;

FIG. 1Q is a front view of the device embodiment of FIG. 1A;

FIG. 1R is a cutaway view of the device of FIG. 1A with a cup attachment;

FIG. 1S is a cutaway view of the head section of the device of FIG. 1A with a cup attachment;

FIG. 1T is an illustration of a block diagram of the electrical components of the device embodiment of FIG. 1 R;

FIG. 1U is an illustration of a block diagram of an exemplary computing/processor apparatus;

FIG. 2A is a top perspective view of a cup attachment embodiment of the present disclosure;

FIG. 2B is a bottom perspective view the cup attachment embodiment of FIG. 2A;

FIG. 2C is s side view of the cup attachment embodiment of FIG. 2A where the other sides are identical;

FIG. 2D is a top view of the cup attachment embodiment of FIG. 2A;

FIG. 2E is a bottom view of the cup attachment embodiment of FIG. 2A;

FIG. 2F is a cutaway of another embodiment where the exterior is illustrated in FIGS. 2A-2E;

FIG. 3A is a top perspective view of another cup attachment embodiment of the present disclosure;

FIG. 3B is a bottom perspective view the cup attachment embodiment of FIG. 3A;

FIG. 3C is s side view of the cup attachment embodiment of FIG. 3A where the other sides are identical;

FIG. 3D is a top view of the cup attachment embodiment of FIG. 3A;

FIG. 3E is a bottom view of the cup attachment embodiment of FIG. 3A;

FIG. 4A is a top perspective view of another cup attachment embodiment of the present disclosure;

FIG. 4B is a bottom perspective view the cup attachment embodiment of FIG. 4A;

FIG. 4C is s side view of the cup attachment embodiment of FIG. 4A where the other sides are identical;

FIG. 4D is a top view of the cup attachment embodiment of FIG. 4A;

FIG. 4E is a bottom view of the cup attachment embodiment of FIG. 4A;

FIG. 5A is a top perspective view of another cup attachment embodiment of the present disclosure;

FIG. 5B is a bottom perspective view the cup attachment embodiment of FIG. 5A;

FIG. 5C is s side view of the cup attachment embodiment of FIG. 5A where the other sides are identical;

FIG. 5D is a top view of the cup attachment embodiment of FIG. 5A;

FIG. 5E is a bottom view of the cup attachment embodiment of FIG. 5A;

FIG. 6A is a top perspective view of another cup attachment embodiment of the present disclosure;

FIG. 6B is a bottom perspective view the cup attachment embodiment of FIG. 6A;

FIG. 6C is s side view of the cup attachment embodiment of FIG. 6A where the other sides are identical;

FIG. 6D is a top view of the cup attachment embodiment of FIG. 6A;

FIG. 6E is a bottom view of the cup attachment embodiment of FIG. 6A;

FIG. 7A is a top perspective view of another cup attachment embodiment of the present disclosure;

FIG. 7B is a bottom perspective view the cup attachment embodiment of FIG. 7A;

FIG. 7C is s side view of the cup attachment embodiment of FIG. 7A;

FIG. 7D is a top view of the cup attachment embodiment of FIG. 7A;

FIG. 7E is a bottom view of the cup attachment embodiment of FIG. 7A;

FIG. 8 is an illustration of a use embodiment of an embodiment of the present disclosure in a facial treatment;

FIG. 9A is an illustration of another use embodiment of an embodiment of the present disclosure in a facial treatment;

FIG. 9B is an illustration of another use embodiment of an embodiment of the present disclosure in a facial treatment;

FIG. 9C is an illustration of another use embodiment of an embodiment of the present disclosure in a facial treatment;

FIG. 9D is an illustration of another use embodiment of an embodiment of the present disclosure in a facial and head treatment;

FIG. 9E is an illustration of another use embodiment of an embodiment of the present disclosure in a facial treatment; and

FIG. 9F is an illustration of another use embodiment of an embodiment of the present disclosure in a facial and neck treatment.

### SUMMARY

In one embodiment, a skin treatment device is provided. The skin treatment device includes a housing including a suction device; a head section including a nozzle, the nozzle connected to the suction device to create a suction force therethrough and including a cup attachment and a light source; and a cup attachment connected to the nozzle and configured to be held against a person's skin in order to apply the suction force thereto.

In another embodiment, a skin treatment device is provided. The skin treatment device includes a housing including a suction device and a power source; a head section including a nozzle, the nozzle connected to the suction device to create a suction force therethrough and including a cup attachment and a light source; and a removable cup attachment connected to the nozzle and configured to be held against a person's skin in order to apply the suction force thereto, the removable cup attachment including a distal aperture configured to be held against the person's skin and moved in a sweeping motion, wherein the power source provides power to the suction device and light source and the light source is positioned such that the light produced from the light source is directed toward the distal aperture.

In another embodiment, a method of using a skin treatment device for treating a user's skin is provided. The skin treatment device includes a housing including a suction device and a power source; a head section including a nozzle, the nozzle connected to the suction device to create a suction force therethrough and including a cup attachment and a light source; and a removable cup attachment connected to the nozzle and configured to be held against a person's skin in order to apply the suction force thereto, the removable cup attachment including a distal aperture is configured to be held against the person's skin and moved in a sweeping motion, wherein the power source provides power to the suction device and light source and the light source is positioned such that the light produced from the light source is directed toward the distal aperture. The method includes holding the distal aperture against the person's skin; activating the suction force and light produced from the light source; moving the distal aperture over the person's skin of the user while maintaining the suction force and light produced from the light source.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention. As used herein, “about” may be understood by persons of ordinary skill in the art and can vary to some extent depending upon the context in which it is used. If there are uses of the term which are not clear to persons of ordinary skill in the art, given the context in which it is used, “about” may mean up to plus or minus 10% of the particular term.

Devices and methods of the present disclosure include embodiments that utilize skin care and other near surface tissue treatments that include applying suction to the surface of a person's or other animal's body including, for example, skin including the skin of the face. The person's or other animal's body surface to which the suction is applied can be a treatment area of the person's or other animal's body.

Suction can be used to create a vacuum on the surface of the skin, any skin, to facilitate absorption. Suction is a non-invasive facial massage technique the helps temporarily bring up the circulation to the skin to helps tighten, brighten and tone and is a non-invasive facial massage technique the helps temporarily bring up the circulation to the skin to helps tighten, brighten and tone and a rush of blood and lymph to the skin from deeper tissues and opens blood vessels near the surface. The skin becomes more receptive to absorb the skin care product than the baseline rate of just applying the skin care product on the skin. No matter the composition or type of skin, there is an increase over the previous rate of absorption without suction. Movement of the suction through a given treatment area of the skin is more efficient and is less likely to leave or leaves minimal unsightly marks on the treatment area as static application of suction where there is no movement of the suction. Suction can also help with exfoliation.

One aspect of the present disclosure is a device, preferably a handheld portable device, that includes and can be used to simultaneously apply during a treatment period (of time) suction and electromagnetic radiation including blue, yellow, green and/or red light to a treatment area of the skin of a user, also including movement of the applied suction and electromagnetic radiation within the treatment area during the treatment period.

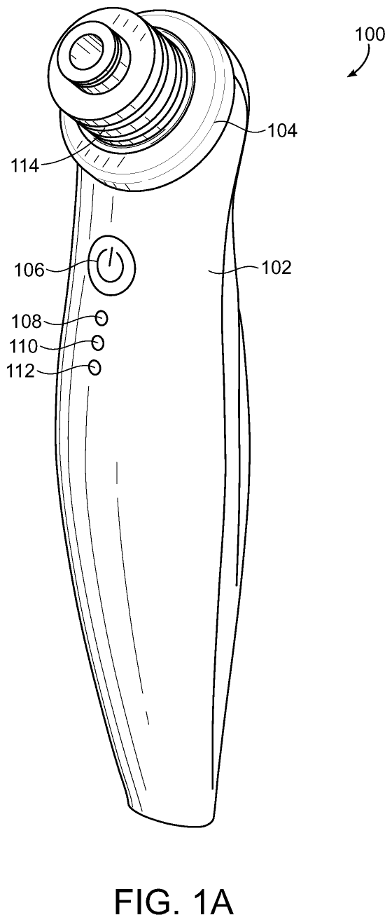

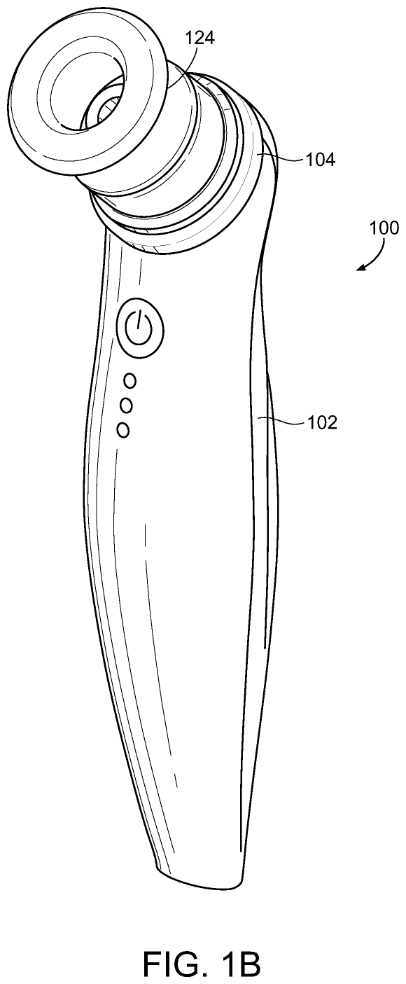

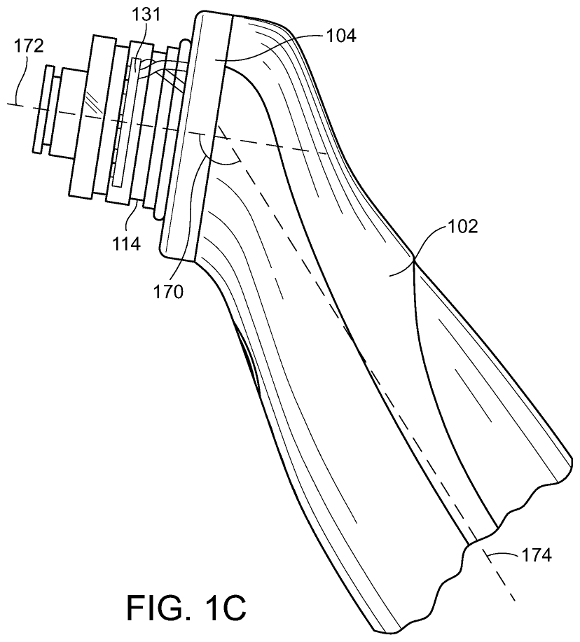

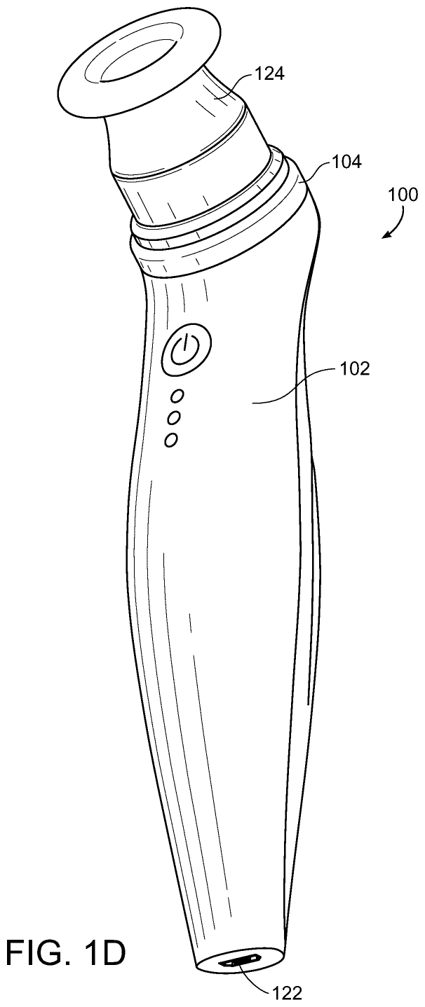

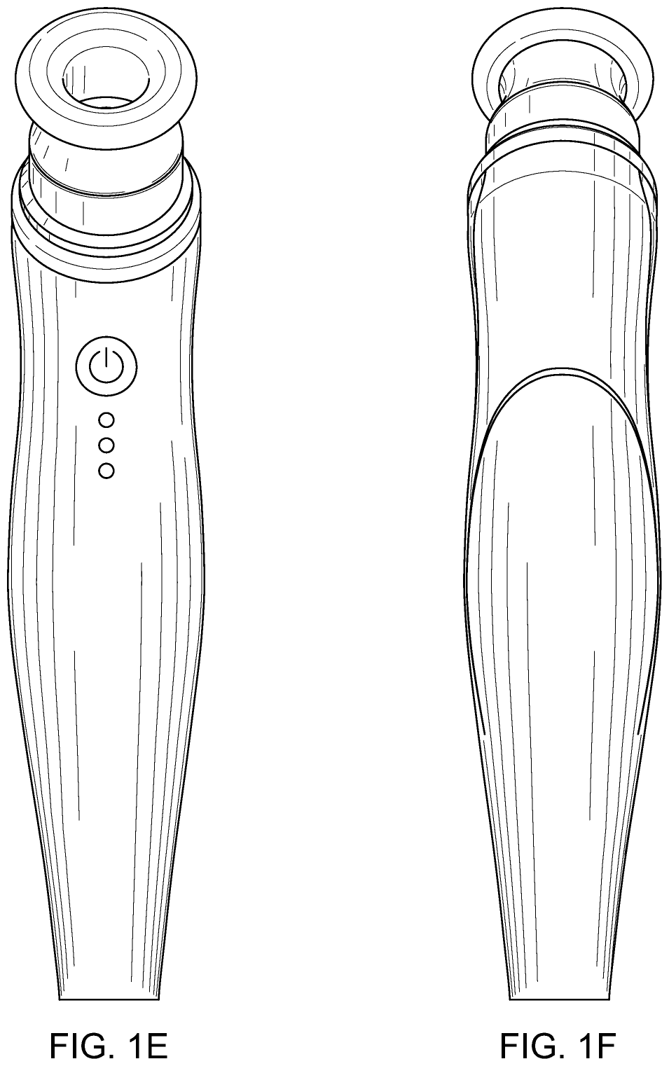

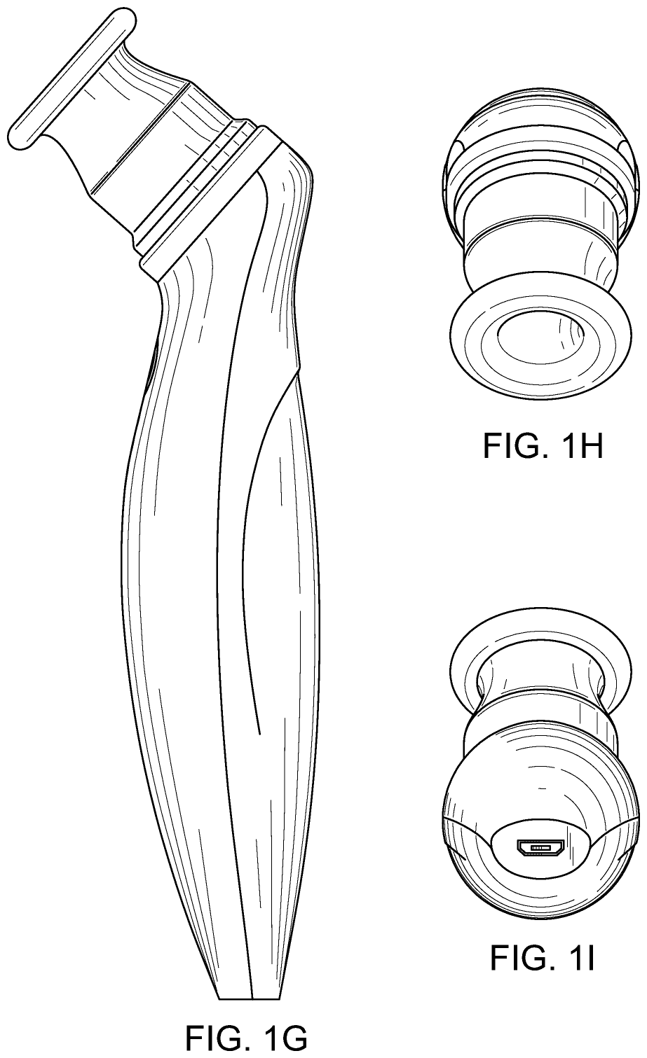

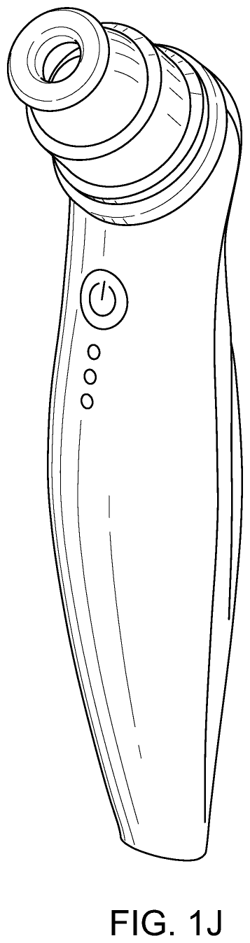

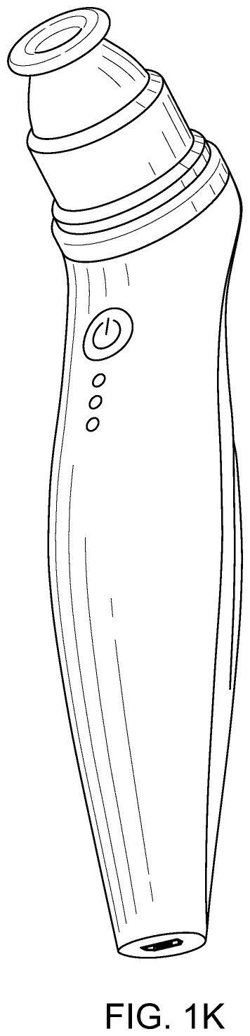

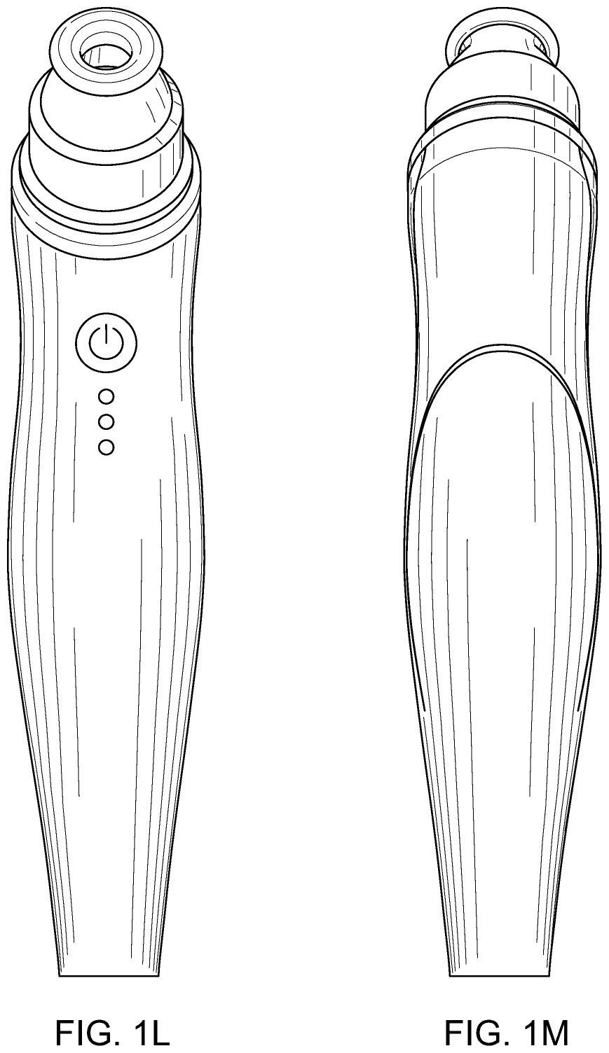

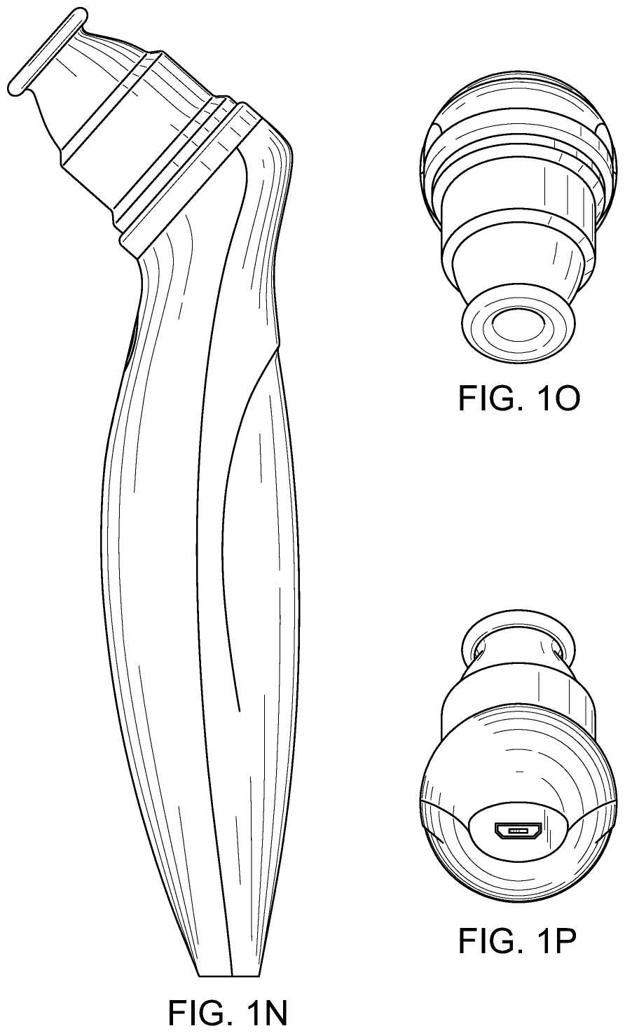

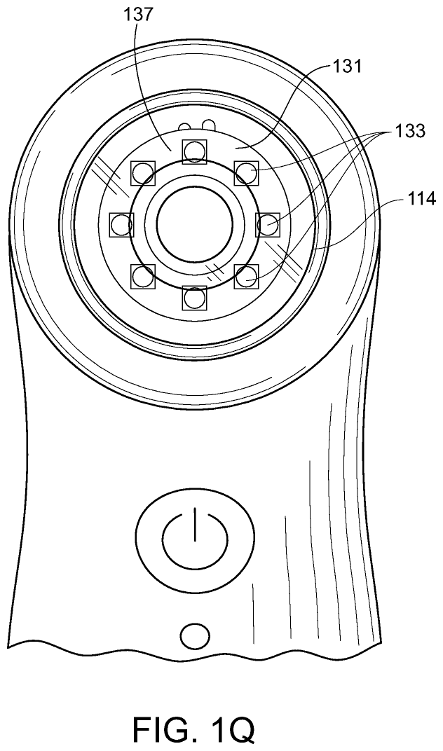

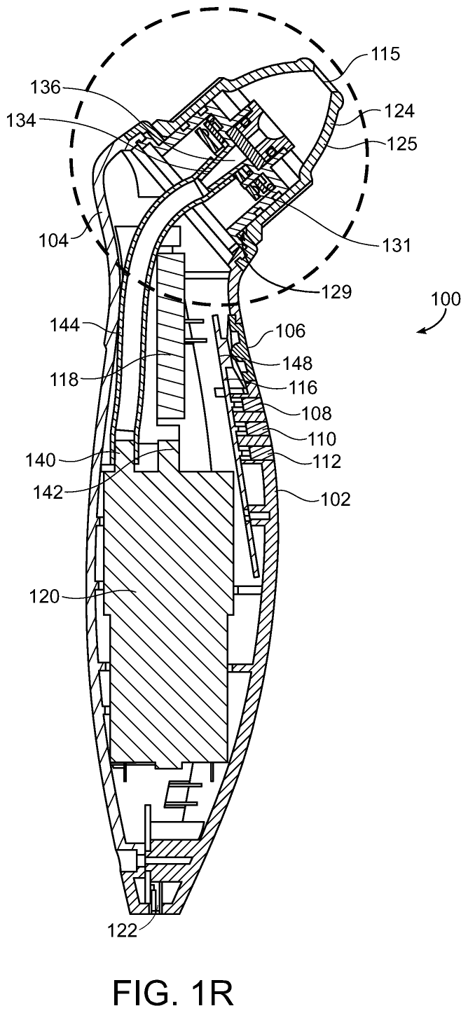

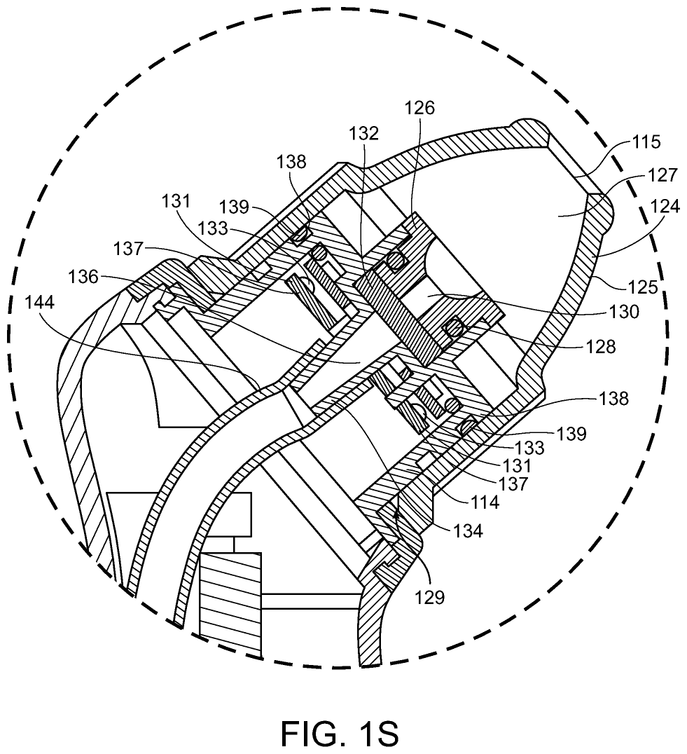

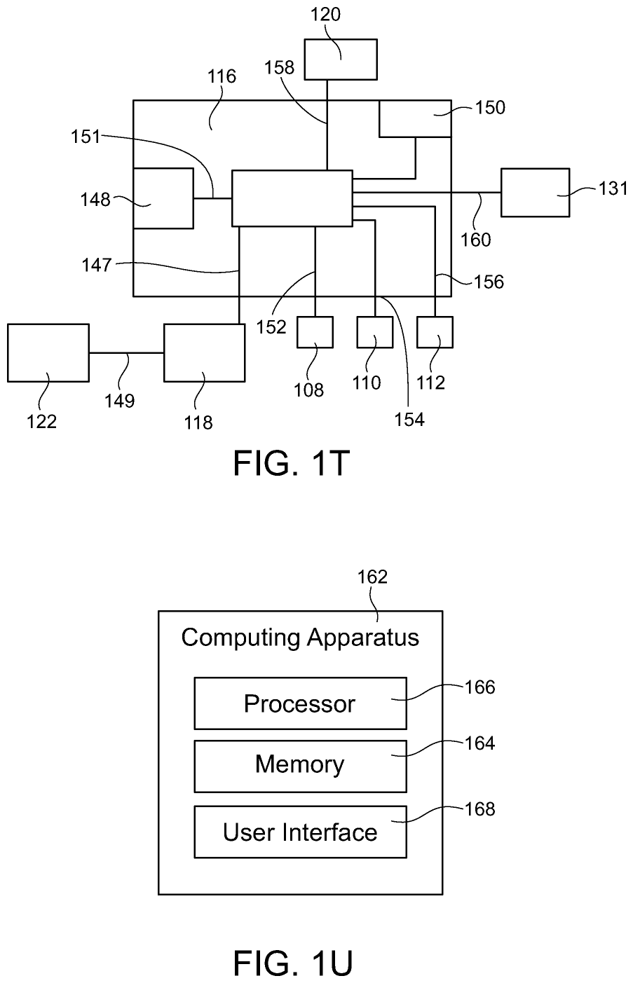

One embodiment of the present disclosure is a device that is a handheld facial treatment device shown in FIGS. 1A-1U. The device 100 includes an elongated housing 102 that can be used to grip the device using, for example, the hand of the user and may be tapered and a head section 104 at one end of the elongated housing 102. The user can be a person self-treating themselves or treating another person. The elongated housing 102 can include an on/off switch 106 and indicator lights 108, 110 and 112 on the exterior. Indicator light 108, 110 and 112 can indicate to the person using the device the current level of suction at which the device is operating. Device 100 includes a housing that is elongated, however, that is exemplary and need not be limited to being elongated in shape (e.g., substantially spherical or oval in shape) as long as it can be held in the hand of a user.

FIGS. 1R and 1S illustrate a cutaway view of exemplary electro-mechanical components included in the device 100 including elongated housing 102 a head section 104. The elongated housing 102 includes on the interior thereof a printed circuit board (PCB) 116, a power source 118 (e.g., a battery including a rechargeable battery) electrically connected to PCB 116 and components thereof, a suction device 120 (e.g., a vacuum pump) for generating a suction force electrically connected to PCB 116 and components thereof and a light source 131 electrically connected to PCB 116 and components thereof. The power source 118 provides electrical power to the suction device 120 and light source 131 and need not be local to the device 100 and can be remote thereto including, for example, a remote power source via, for example, power connector 122 electrically connected to the power source 118 that can be connected to for example, a remote battery including a rechargeable battery, an electrical outlet, a cellphone, a computer, etc. though the use of a variety of electrical conduits and connectors (two-prong plug, three-prong plug, USB, etc.). Power connector 122 can also be used to recharge power source 118 where it is a power source, for example, can be rechargeable battery and, thus, device 100 can be operated as a cordlessly when the rechargeable battery includes electrical energy to provide electrical power to the suction device 120 and light source 131. The on/off switch 106 is positioned adjacent to so as to interact with switch 148 on PCB 116 and components thereof and indicator lights 108, 110 and 112 are also electrically connected to PCB 116 and components thereof.

The head section 104 includes a nozzle including nozzle cup attachment 114 that is used to connect a cup attachment including one of the removable cup attachment embodiments of the present disclosure 124 to the head section 104. Removable cup attachment embodiments 124 can include a body 125 with an interior channel 127, a proximal aperture 129 through which the removable cup attachment is connected the nozzle including nozzle cup attachment 114 and a distal aperture 115 such that the end of the removable cup attachment embodiment 124 with distal aperture 115 can be placed in contact with the skin of the user during use. The nozzle including nozzle cup attachment 114 includes a suction head 126 including a sealing ring 128 and a head suction channel 130, a filter sponge 132 made of a suitable material (e.g., fabric) to prevent foreign material from passing further into the device 100, a suction nozzle 134 including a nozzle suction channel 136 and a annular slot 138 into which a cup annular sealing ring 139 of the removable cup attachment embodiment 124 in inserted upon attachment. The suction device 120 includes an air inlet 140 and air outlet 142. Air inlet 140 is connected to the nozzle suction channel 136 via suction hose 144.

A light source 131 is also included in the nozzle including nozzle cup attachment 114 that includes light generating elements 133, preferably in a substantially circular arrangement with the light generating elements 133 directing their light in the direction of the distal aperture 115 such that the light is directed at the skin of the user of the device when the distal aperture 115 is placed in contact with the skin of the user during use. The nozzle including nozzle cup attachment 114 is substantially optically clear so that the light from the light source 131 can pass therefrom and toward the skin of the user during use. The light source can include a plurality of light generating elements 133, e.g., LEDs, on a printed circuit board (PCB) 137 that are wired to receive power from a power source power source 118 (e.g., a battery including a rechargeable battery) via PCB 116 and components thereof in the elongated housing 102. The light generating elements 133 can number from about 4 to as many as about 16 or more (preferably about 8) that are substantially evenly spaced around the substantially circular shape of the PCB 116 so as to provide substantially even application of light to the skin of the user to which suction is applied through the distal aperture 115 when is placed in contact with the skin of the user during use. The various removable cup attachments of the present disclosure and will be described in more detail subsequently.

An exemplary block diagram shows the electro-mechanical components of device 100 and connections therebetween is illustrated in FIG. 1T. PCB 116 includes a controller 146 that is connected to power source 118 via electrical conduit 147, switch 148 via electrical conduit 151, indicator lights 108, 110 and 112 via electrical conduits 152, 154 and 156 respectively, suction device 120 via electrical conduit 158 and light source 131 via electrical conduit 160. Power source 118 is connected to power connector 122 via electrical conduit 149

PCB 116 may optionally include a vibration generating device 150 which can be, for example, a micro-vibration motor which has an offset impeller to make the whole device 100 vibrate. The vibrations generated by the vibration generating device 150 can provide a messaging action to the area the removable cup attachment that is in contact with the skin of the user and can be used while the light source is active or inactive. The micro vibrations can be at a frequency ranging from about 300 Hz to about 350 Hz, about 325 Hz. Vibration generating device 150 can also be remote from PCB 116, but local to the device 100, e.g., the interior of device 100, as long as it is capable of making the whole device 100 vibrate. Such vibrations can provide massaging action to the skin of the user where the device is being applied, such massaging action can increase blood flow to that skin area and other adjacent tissues.

In at least one aspect of the disclosed embodiments, the systems and methods disclosed herein may be executed by the controller 146 which may include, for example, electronic circuitry or one or more computers or processor-based components (e.g., CPU/controller) under the control of one or more programs stored on computer readable medium, such as a non-transitory computer readable medium. FIG. 1U shows a block diagram of an exemplary computing apparatus 162 that may be used to practice aspects of the disclosed embodiment. In at least one exemplary aspect, the digital receiver/processor and other disclosed devices, components and systems may be implemented using an instance or replica of the computing apparatus 162 or may be combined or distributed among any number of instances or replicas of computing apparatus 162.

A computing apparatus 162 may include computer readable program code or machine readable executable instructions (such as, for example, instructions to pump fluids and operate the agitator motors utilized in the embodiments disclosed herein) stored on at least one computer readable medium 164, which when executed, are configured to carry out and execute the processes and methods described herein, including all or part of the embodiments of the present disclosure. The computer readable medium 164 may be a memory of the computing apparatus 162. In alternate aspects, the computer readable program code may be stored in a memory external to, or remote from, the apparatus 162. The memory 164 may include magnetic media, semiconductor media, optical media, or any media which may be readable and executable by a computer. Computing apparatus 162 may also include a processor 166 for executing the computer readable program code stored on the at least one computer readable medium 164. In at least one aspect, computing apparatus 162 may include one or more input or output devices to allow communication among the components of and those connected to the system model, including, for example, operation of the light source 131 and vibration generating device 150 using what may be generally referred to as a user interface 168, which may be connected to or operate as switch 148, which may operate the other components included in or connected to CPU/controller 146 or to provide input to the computing apparatus 162 to or from other components of the system model.

Device 100 can be operated, for example, by the user pressing on/off button 106 which activates via switch 148, controller 146 and related components of the present disclosure, the suction device 120 (thereby causing a suction force at the distal aperture 115), the light source 131 and turning on one of the indicator light 108, 110 and 112 as a first operational stage. Further exemplary operation can involve the user pressing the on/off button 106 again to change via switch 148, controller 146 and related components of the present disclosure, the controller 146 sending a signal to change the amount of suction exerted by the suction device 120 to make it more or less than the amount of suction previously while maintaining the light source 131 and turning on a different indicator light 108, 110 and 112 as a second operational stage. Further exemplary operation can involve the user pressing the on/off button 106 still again to change via switch 148, controller 146 and related components of the present disclosure, the controller 146 sending a signal to change the amount of suction exerted by the suction device 120 to make it more or less than the amount of suction of previously operation stages while maintaining the light source 131 and turning on a different indicator light 108, 110 and 112. Still further exemplary operation can involve the user pressing the on/off button 106 a fourth time to turn off via the switch 148 and controller 146 the suction device 120, the light source 131 and the indicator light 108, 110 and 112 activated in the previous step. The number of operational stages is not limited nor are the number of indicator lights. In embodiments that include a vibration generating device 150 which is an optional component, the vibration generating device 150 can be activated via the controller 146 to provide a vibrating effect during any one or all the operational stages (e.g., the first pressing of the on/off button 106, the first pressing of the on/off button 106, etc.) when both suction and light are activated. The vibration generating device 150 can have the same amount of vibration for the operational stages or different amounts of vibration for each operational stage.

The amount of suction caused by the suction device 120 during operation stages can be one amount suction force or a plurality of various suction forces. Each suction force should provide a gentle suction force to the skin, but not so strong as to damage or cause disfigurement thereof. In this exemplified embodiment, the amounts of suction can be in escalating order or descending order or another order from the first pressing of the on/off button 106 to the last pressing of on/off button 106 that turns off the device.

The angle 170 of the head section 104 along its length wise 172 relative to the length wise axis 174 of the elongated housing can vary. Preferably, that angle 170 can be along the length wise axis 174 of the elongated housing at about 0° to about 90° relative to the length wise axis 174 of the elongated housing or any angle in between, as shown in the illustration of FIG. 10.

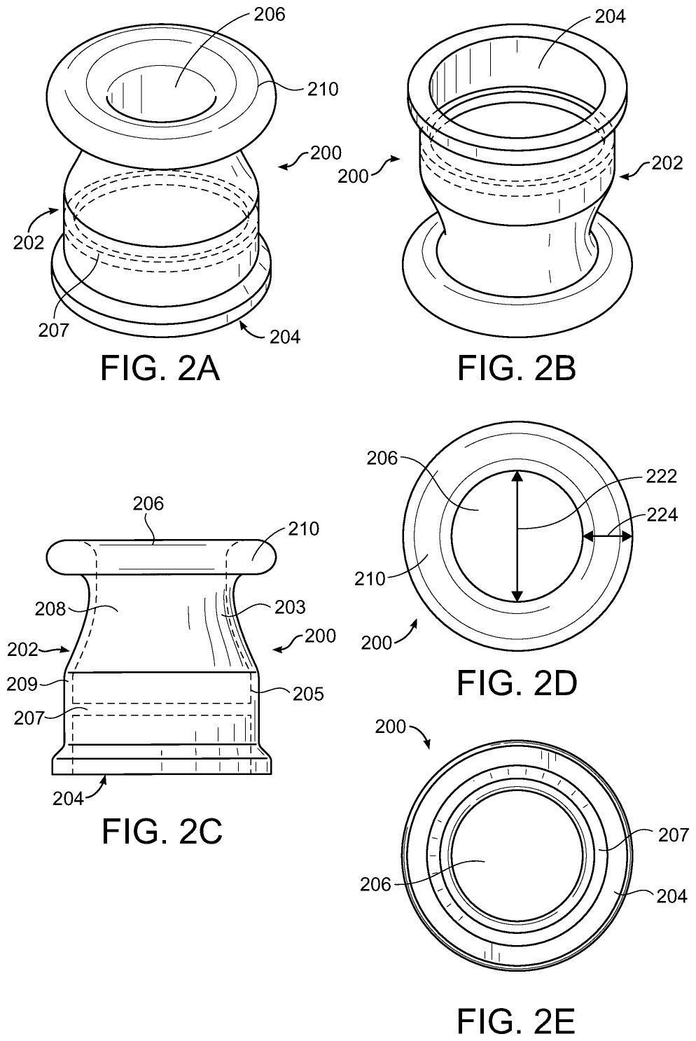

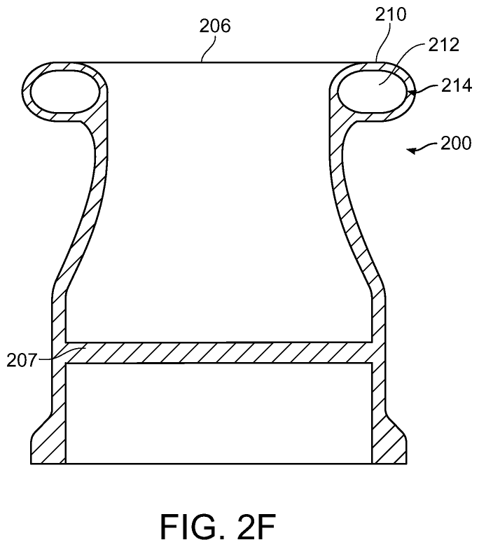

The removable cup attachment embodiments of the present disclosure can take various forms and shapes (such as, for example conical or bell shaped) made of rigid or semi-rigid (including flexible semi-rigid) materials and components such as silicone, plastic (preferably BPA free plastic), glass or a combination thereof. Exemplary embodiments of the cup, as shown in FIGS. 2A-2F, 3A-3E, 4A-4E, 5A-5E, 6A-6E and 7A-7E. Removable cup attachment 200 in FIG. 2A-2E includes a body 202 that is generally circular in cross-section with a concave tapered body section 203 and a cylindrical body section 205, a proximal aperture 204 generally circular in cross-section, a distal aperture 206 generally circular in cross-section, an interior channel 208 between proximal aperture 204 and distal aperture 206, a cup annular sealing ring 207 on the interior wall 209 of removable cup attachment 200 that can be inserted, for example, in FIGS. 1R and 1S into annular slot 138 in the nozzle including nozzle cup attachment 114 to secure removable cup attachment 200 thereto and a rim 210 adjacent distal aperture 206. The diameter 222 of the distal aperture 206 can range in size from about 0.5 cm to about 2 cm, about 1.5 cm and the width 224 of the rim 210 can range in size from about 0.5 cm to about 1.5 cm, about 1 cm.

Another embodiment of the removable cup attachment illustrated in FIGS. 2A-2F (FIGS. 2A-2E show the exterior or the embodiment and FIG. 2F shows the interior) where the embodiment of FIGS. 2A-2E may optionally include a phase change material 212 positioned, for example, in an annular space 214 on the interior of rim 210 as shown in FIG. 2F. As a result of including the phase change material, the cup attachment and the phase change material included therein can be heated (e.g., by placing it in hot water or heating it in a microwave or other oven) or cooled (e.g., by placing it in a refrigerator or freezer) in order to provide heat or cold to the skin and adjacent tissues of the user where the device is being applied. The heating or cooling of the area can have therapeutic benefits. For example, cooling of the area can result in vasoconstriction, thereby reducing blood flow, that can aid in certain medical conditions including, reducing puffy skin (including, e.g., around the eyes), lessoning headaches and reducing the swelling of tissues.

The phase change material 212 can include various water/alcohol/polymer gel or semi-solid materials including, for example, a glycerin and polyacrylamide based material and water that has thermal properties to allow the gel to be either heated or cooled and a material sold under the trademark (ELASTO GEL) by Southwest Technologies, Inc. of Kansas City, Mo. Such gel compositions useful as phase change materials can also include gel compositions disclosed in U.S. Pat. No. 4,462,224 in col. 1, lines 45-55 and U.S. Pat. No. 4,736,311 in col. 3, lines 10-39, which disclosures are herein incorporated by reference. A method embodiment of the present disclosure can include cooling or heating the cup attachment including a phase change material as described herein and engaging in one of the treatment method embodiments of the present disclosure operating device embodiments including cup attachment embodiments of the present disclosure.

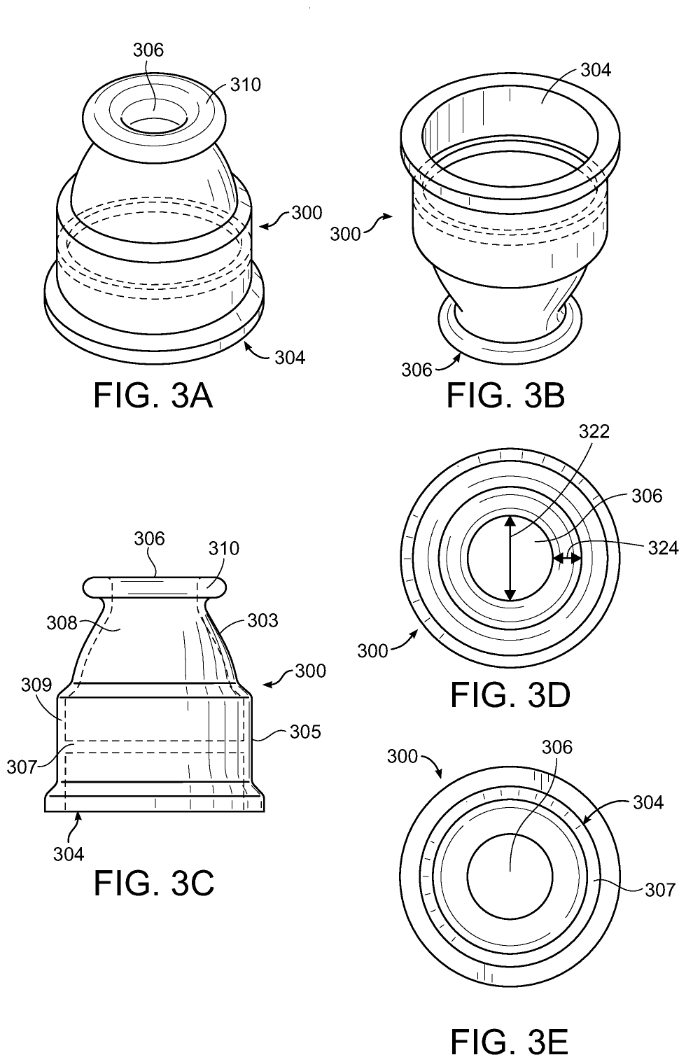

Cup attachment 300 in FIG. 3A to 3E includes a body 300 that is generally circular in cross-section with a convex tapered body section 303 and a cylindrical body section 305, a proximal aperture 304 generally circular in cross-section, a distal aperture 306 generally circular in cross-section, an interior channel 308 between proximal aperture 304 and distal aperture 306, a cup annular sealing ring 307 on the interior wall 309 of cup attachment 300 that can be inserted, for example, in FIGS. 1R and 1S into annular slot 138 in the nozzle including nozzle cup attachment 114 to secure cup attachment 300 thereto and a rim 310 adjacent distal aperture 306. The diameter 322 of the distal aperture 306 can range in size from about 0.5 cm to about 2 cm, about 1.5 cm and the width 324 of the rim 310 can range in size from about 0.5 cm to about 1.5 cm, about 1 cm.

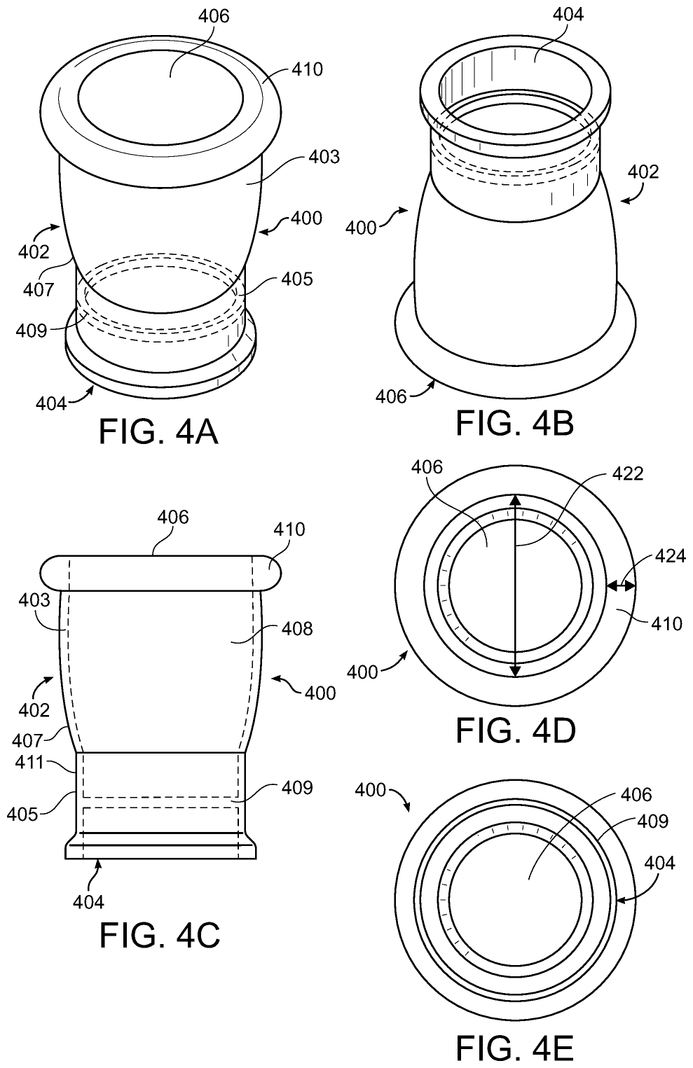

Cup attachment 400 in FIG. 4A to 4E includes a body 402 that is generally circular in cross-section with a cylindrical body section 405 and a bell shaped portion 403 with a rounded portion 407 adjacent the cylindrical body section 405 404 generally circular in cross-section, a distal aperture 406 generally circular in cross-section, an interior channel 408 between proximal aperture 404 and distal aperture 406, a cup annular sealing ring 409 on the interior wall 411 of cup attachment 400 that can be inserted, for example, in FIGS. 1R and 1S into annular slot 138 in the nozzle including nozzle cup attachment 114 to secure cup attachment 400 thereto and a rim 410 adjacent distal aperture 406. The diameter 422 of the distal aperture 406 can range in size from about 0.5 cm to about 3 cm, about 2 cm and the width 424 of the rim 410 can range in size from about 0.5 cm to about 1.5 cm, about 1 cm.

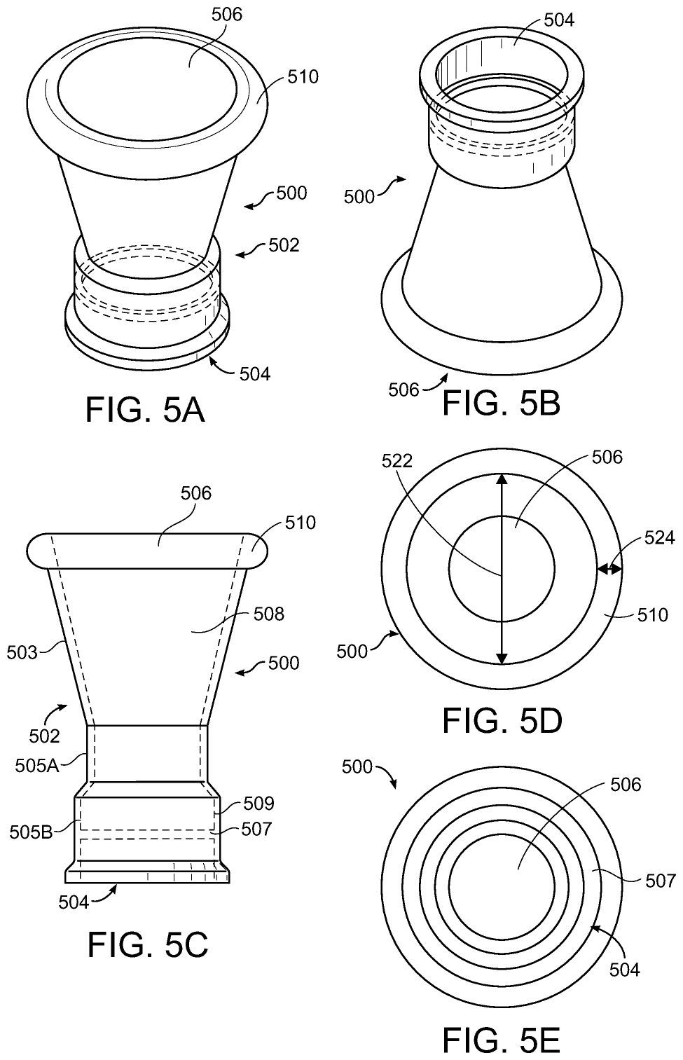

Cup attachment 500 in FIG. 5A to 5E includes a body 502 that is generally circular in cross-section with a conical shaped portion 503, a first cylindrical body section 505A and a second cylindrical body section 505B, a proximal aperture 504 generally circular in cross-section, a distal aperture 506 generally circular in cross-section, an interior channel 508 between proximal aperture 504 and distal aperture 506, a cup annular sealing ring 507 on the interior wall 509 of cup attachment 500 that can be inserted, for example, in FIGS. 1R and 1S into annular slot 138 in the nozzle including nozzle cup attachment 114 to secure cup attachment 500 thereto and a rim 510 adjacent distal aperture 506. The diameter 522 of the distal aperture 506 can range in size from about 0.5 cm to about 3 cm, about 2 cm and the width 524 of the rim 510 can range in size from about 0.5 cm to about 1.5 cm, about 1 cm.

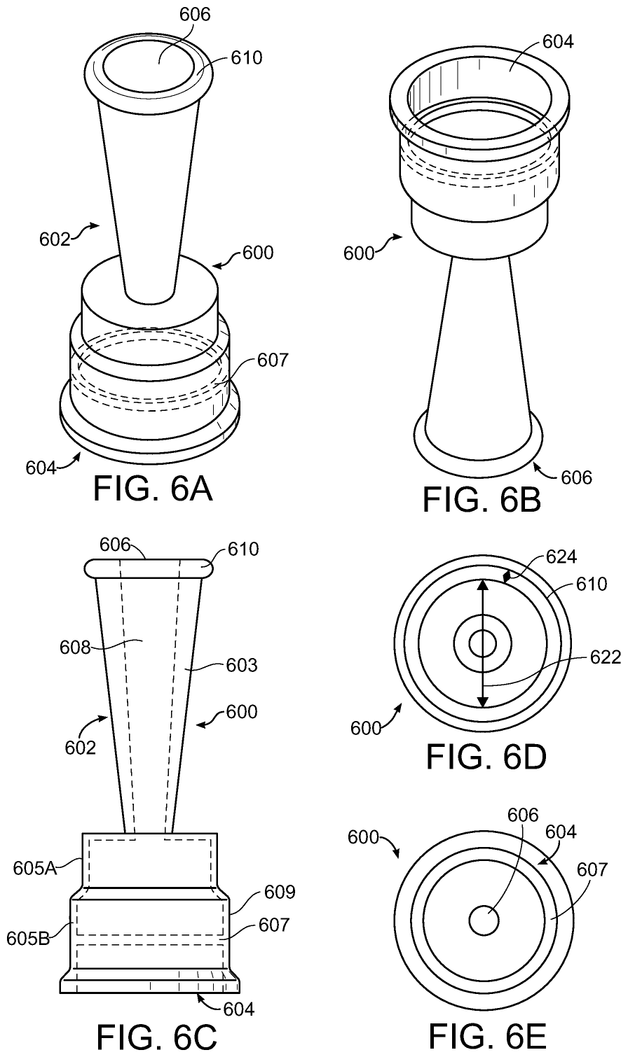

Cup attachment 600 in FIG. 6A to 6E includes a body 602 that is generally circular in cross-section with a conical shaped portion 603, a first cylindrical body section 605A and a second cylindrical body section 605B, a proximal aperture 604 generally circular in cross-section, a distal aperture 606 generally circular in cross-section, an interior channel 608 between proximal aperture 604 and distal aperture 606, a cup annular sealing ring 607 on the interior wall 609 of cup attachment 600 that can be inserted, for example, in FIGS. 1R and 1S into annular slot 138 in the nozzle including nozzle cup attachment 114 to secure cup attachment 600 thereto and a rim 610 adjacent distal aperture 606. The diameter 622 of the distal aperture 606 can range in size from about 0.1 cm to about 0.5 cm, about 0.3 cm and the width 624 of the rim 610 can range in size from about 0.1 cm to about 0.5 cm, about 0.3 cm. Cup attachment 600 can be used in the eye area and other delicate areas.

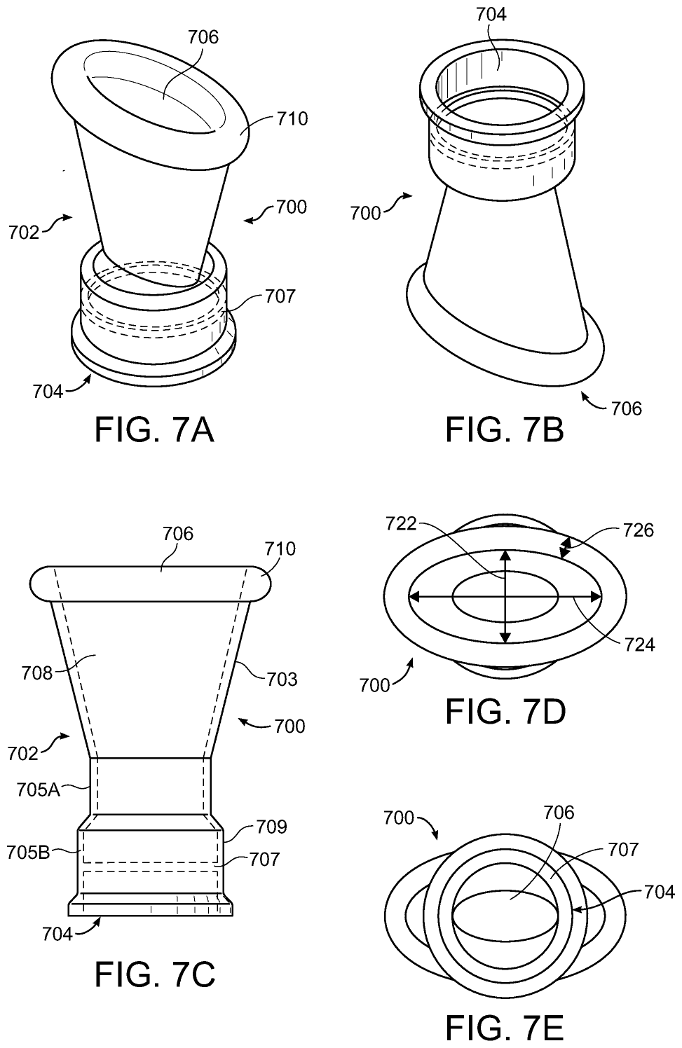

Cup attachment 700 in FIG. 7A to 7E includes a body 702 with a conical shaped portion 703 that is generally oval in cross-section, a first cylindrical body section 705A and a second cylindrical body section 705B, a proximal aperture 704 generally circular in cross-section, a distal aperture 706 generally oval in cross-section, an interior channel 708 between proximal aperture 704 and distal aperture 706, a cup annular sealing ring 707 on the interior wall 709 of cup attachment 700 that can be inserted, for example, in

FIGS. 1R and 1S into annular slot 138 in the nozzle including nozzle cup attachment 114 to secure cup attachment 700 thereto and a rim 710 adjacent distal aperture 706. The distance 722 of the distal aperture 706 can range in size from about 0.5 cm to about 2 cm, about 1 cm, distance 724 of the distal aperture 706 can range in size from about 1 cm to about 4 cm, about 3 cm and the width 726 of the rim 710 can range in size from about 0.5 cm to about 1.5 cm, about 1 cm.

During use, the distal apertures and adjacent rim sections of the cup embodiments are placed in contact with the skin surface of the treatment area.

The light source 131 that includes a plurality of light generating elements 133 adds further benefits to the treatment process and embodiments of the present disclosure. The simultaneous light treatment can be directed toward the treatment area of the user, preferably passing through the interior channel the cup attachment. The light treatment can include continuous or pulsed electromagnetic radiation including blue, yellow, green and/or red light, preferably red and/or near infrared light, in wavelengths of, for example, between about 560 nm to about 1200 nm, about 560 nm and about 980 nm, about 630 nm to about 660 nm, about 630 nm to about 850 nm including a yellow wavelength of about 590 nm to about 560 nm, a near-infrared wavelength of about 610 nm to about 980 nm and a red wavelength of about 700 nm to about 635 nm including about 660 nm to produce fast relief and healing with minimal, if any, side effects, about 660 nm being more readily absorbed. Red and near-infrared light can penetrate tissue because these wavelengths are not blocked by blood or water as much as other wavelengths. These wavelengths can be used in medical settings for burn treatment, wound healing, joint and muscle pain, sports injuries, etc. Light treatment using embodiments of the present disclosure can also be used in the near infrared, for example, in the range of from about 800 nm to about 1200 nm, about 800 nm to about 900 nm or a wavelength of about 850 nm on the skin of a user's body for muscle pain and/or general pain relief because of the deep penetration of these wavelengths as well as other use embodiments of the present disclosure. Photonic red-light therapy is beneficial because the body's tissue can absorb this type of light at a cellular level, converting it to cellular energy (known as ATP). Studies have shown that this energy can accelerate activity in cellular mitochondria (the part of the cell that generates protein, collagen, and healing materials) as well as improve blood flow in the area being treated, speeding healing and minimizing pain. As a result, methods of the present disclosure using device embodiments of the present disclosure including application of red light can reduce blood flow, have anti-inflammatory effects, tighten the skin and improve skin texture and tone.

Such handheld devices are not cost prohibitive for the average person. Also, over the past several years, new technology allowed improved battery efficiency, increased voltages, and more light output to be packaged into a small affordable device.

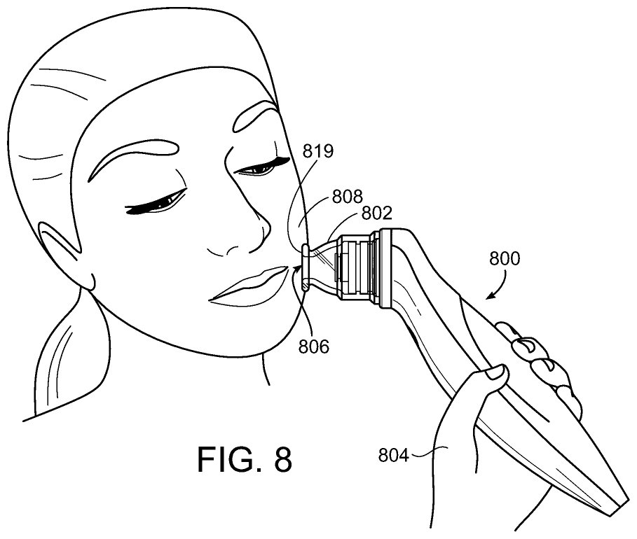

In use, methods of using device embodiments including cup attachment embodiment of the present disclosure are illustrated in FIG. 8. The device 800 with the cup attachment 802, is held in the user's hand 804 and the distal aperture 806 is placed in contact with the skin 808 of the user either before or after suction and the light source is turned on. Next, the distal aperture 806 and rim surface 819 around the distal aperture of cup attachment 802 is moved around the skin of the user 808 in a sweeping motion (in FIG. 8, the face of the user) while maintaining contact of the distal aperture 806 and rim surface 819 with the user's skin 808 and maintaining the suction force, preferably a gentle suction force leaving minimal or no marks behind on the treatment area. Ideally the skin is cleaned and can be prepared to receive the suction cup with an oil-based serum, moisturizer or oil itself being applied to the rim surface 819 around the distal aperture 806 of the cup attachment 802 and applied to the user's skin to be treated before or after suction is applied (preferably before) or to the user's skin 808 that will be in contact with the distal aperture 806 and rim surface 819 around the distal aperture, preferably the latter, to help the cup attachment glide seamlessly and no or minimal abrasion to the skin.

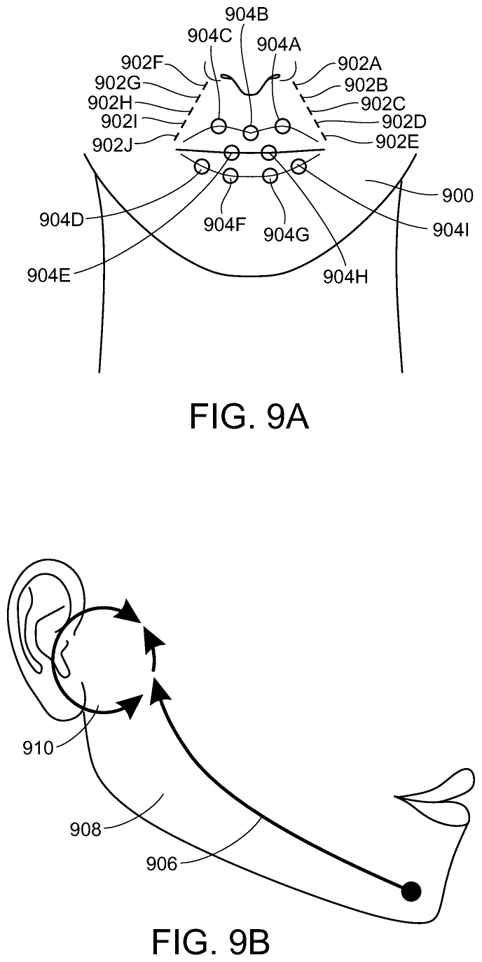

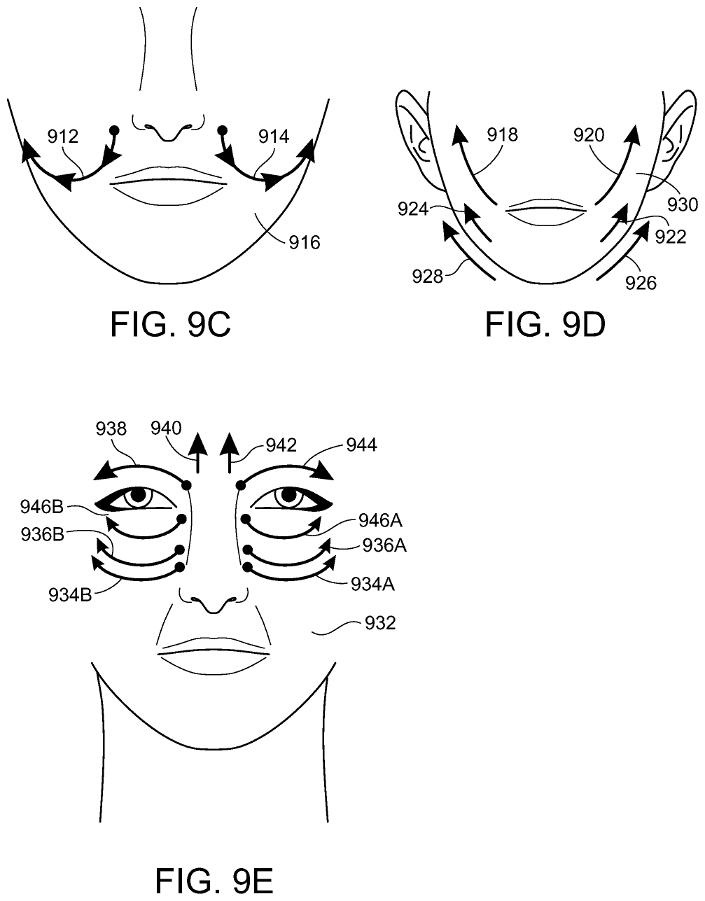

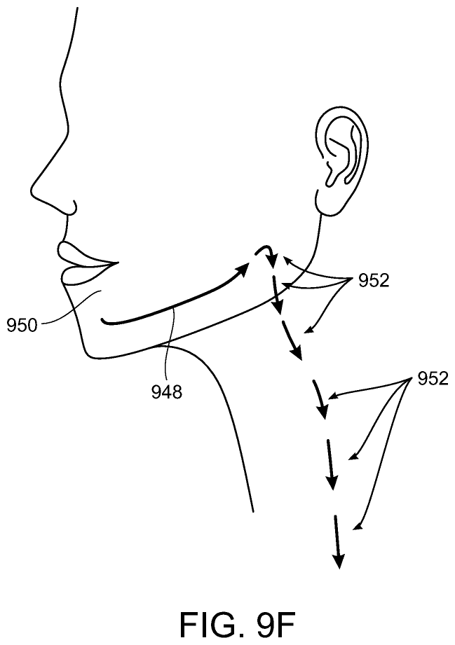

FIGS. 9A-9F illustrate methods of the present disclosure in which exemplary movement pattern for using the methods of the present disclosure for using device embodiments including cup attachment embodiments of the present disclosure. FIG. 9A illustrates an embodiment of using device embodiments including cup attachment embodiments of the present disclosure for face 900 with press and release applications to plump nose to mouth lines at one or more of approximate positions 902A, 902B, 902C, 902D, 902E, 902F, 902G, 902H, 902I, 902J on face 900 and with press and release applications to plump lips at one or more of approximate positions 904A, 904B, 904C, 904D, 904E, 904F, 904G, 904H and 904I on face 900 FIG. 9B illustrates an embodiment of using device embodiments including cup attachment embodiments of the present disclosure for relief of temporomandibular joint (TMJ) and facial tension in which approximate sweeping movement 906 on face 908 can be used for relief of both TMJ and facial tension and approximate circular movement 910 can be used in an area of TMJ. FIG. 9C illustrates an embodiment of using device embodiments including cup attachment embodiments of the present disclosure for the treatment nose and mouth lines in which there is approximate sweeping movements 912 and 914 on face 916. FIG. 9D illustrates an embodiment of using device embodiments including cup attachment embodiments of the present disclosure for contouring cheek bone areas in which approximate sweeping movements 918, 920, 922, 924, 926 and 928 on face 930 and with approximate movements 922, 924, 926 and 928 along the jaw line of face 930. FIG. 9E illustrates an embodiment of using device embodiments including cup attachment embodiments of the present disclosure for face 932 with approximate movements 934A, 936A, 934B and 936B for cheek lift, approximate movements 938, 940, 942, 944 and 946 for sinus pressure and congestion relief and approximate movement 946A and 946B also for reducing under eye puffiness. FIG. 9F illustrates an embodiment of using device embodiments including cup attachment embodiments of the present disclosure for lymphatic drainage in which approximate sweeping movement 948 on face 950 followed by approximate sweeping movement 952 down the side of the jaw and down the side of the neck.

The device and method embodiments of the present disclosure are not limited only to the face, head and neck area, but can be used on any part of the body of the user, the user being human or other animal to help relive muscle soreness and/or tension, including from temporomandibular joint (TMJ) or temporomandibular disorder (TMD) the cupping machine can be moved over the masseter, temporalis on the face and even the sternocleidomastoid (scm) muscle in the neck. TMJ pain can be referred to the neck and upper back as well and can be the causes of headaches. Other benefits can include reducing pain and swelling, increased lymphatic activity, strengthening the immune system, accelerating cell regeneration and increased circulation. Moving the cup continuously over an area of the face and or body can not only relive tension but also to create lymphatic drainage, reduce facial puffiness, tone the skin, increase circulation, reduce fine lines and create a plumpness to the skin. As a result of the increased circulation under the skin surface the skin will be more receptive and or absorptive to any skin care massaged into the face, neck or body.

The device and method embodiments of the present disclosure can also be used to sucks up dead skin cells from the skin areas to which it is applied. Thus, methods of the present disclosure can include the removal of dead skin cells. Thus, one reason for the presence of filter sponge 132 is to capture the dead skin cells so removed and methods of the present disclosure can also include periodically replacing the filter during or between uses so as not to become clogged and block the suction force during treatment.

Another embodiment is a kit that includes an embodiment of the present disclosure and may also include instructions for use and may also include a plurality of cup attachments. Another embodiment is a kit that includes an embodiment of the present disclosure and an oil-based serum, moisturizer or oil itself to help the cup glide seamlessly and no abrasion to the skin and may also include instructions for use and may also include a plurality of cup attachments.

The methods of the present disclosure also include the treatments, uses and device operations included herein.

While there have been shown, described and pointed out, fundamental features of the present disclosure as applied to the exemplary embodiments thereof, it will be understood that various omissions and substitutions and changes in the form and details of compositions, devices and methods illustrated, and in their operation, may be made by those skilled in the art without departing from the spirit or scope of the present disclosure. Moreover, it is expressly intended that all combinations of those elements and/or method steps, which perform substantially the same function in substantially the same way to achieve the same results, are within the scope of the present disclosure. Moreover, it should be recognized that structures and/or elements and/or method steps shown and/or described in connection with any disclosed form or embodiment of the present disclosure may be incorporated in any other disclosed or described or suggested form or embodiment as a general matter of design choice. It is the intention, therefore, to be limited only as indicated by the scope of the claims appended hereto.

This written description uses examples as part of the disclosure, including the best mode, and also to enable any person skilled in the art to practice the disclosed implementations, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A skin treatment device, comprising:
a housing including a suction device;
a head section including a nozzle, the nozzle connected to the suction device to create a suction force therethrough and including a cup attachment and a light source; and
a cup attachment connected to the nozzle and configured to be held against a person's skin in order to apply the suction force thereto.

2. The skin treatment device of claim 1, wherein the cup attachment is made of silicone, plastic, glass or a combination thereof.

3. The skin treatment device of claim 1, wherein the cup attachment is removable from the nozzle.

4. The skin treatment device of claim 1, wherein the cup attachment is configured to be held against the skin and moved in a sweeping motion.

5. The skin treatment device of claim 1, wherein the light source generates electromagnetic radiation including at least one of red, blue, purple yellow and or green light.

6. The skin treatment device of claim 1, wherein the light source generates electromagnetic radiation in the range of from about 630 nm to about 850 nm.

7. A skin treatment device, comprising:
a housing including a suction device and a power source;
a head section including a nozzle, the nozzle connected to the suction device to create a suction force therethrough and including a cup attachment and a light source; and
a removable cup attachment connected to the nozzle and configured to be held against a person's skin in order to apply the suction force thereto, the removable cup attachment including a distal aperture configured to be held against the person's skin and moved in a sweeping motion,
wherein the power source provides power to the suction device and light source and the light source is positioned such that the light produced from the light source is directed toward the distal aperture.

8. The skin treatment device of claim 7, wherein the power source is a rechargeable battery and the skin treatment device can be operated cordlessly.

9. The skin treatment device of claim 7, wherein the cup attachment is made of silicone, plastic, glass or a combination thereof.

10. The skin treatment device of claim 7, wherein the cup attachment is removable from the nozzle.

11. The skin treatment device of claim 7, wherein the distal aperture of the cup attachment is configured to be held against the skin and moved in a sweeping motion.

12. The skin treatment device of claim 7, wherein the light source is an LED light source generating electromagnetic radiation including at least one of red, blue, purple yellow and or green light.

13. The skin treatment device of claim 7, wherein the light source an LED light source that generates electromagnetic radiation in the range of from about 630 nm to about 850 nm.

14. A method of using a skin treatment device for treating a user's skin, the skin treatment device comprising:
a housing including a suction device and a power source;
a head section including a nozzle, the nozzle connected to the suction device to create a suction force there through and including a cup attachment and a light source; and
a removable cup attachment connected to the nozzle and configured to be held against a person's skin in order to apply the suction force thereto, the removable cup attachment including a distal aperture is configured to be held against the person's skin and moved in a sweeping motion,
wherein the power source provides power to the suction device and light source and the light source is positioned such that the light produced from the light source is directed toward the distal aperture, the method comprising:
holding the distal aperture against the person's skin;
activating the suction force and light produced from the light source; and
moving the distal aperture over the person's skin of the user while maintaining the suction force and light produced from the light source.

15. The method of claim 14, wherein the power source is a rechargeable battery and the skin treatment device can be operated cordlessly.

16. The method of claim 14, wherein the cup attachment is made of silicone, plastic, glass or a combination thereof.

17. The method of claim 14, wherein the cup attachment is removable from the nozzle.

18. The method of claim 14, further including administering an oil-based serum, moisturizer or oil to an area of the person's skin prior to holding the distal aperture against it and placing positioning the distal aperture against the area of the person's skin to which the serum or oil was administered.

19. The method of claim 14, wherein the method is used to treat temporomandibular joint (TMJ) or temporomandibular disorder (TMD) facial tension.

20. The method of claim 14, wherein the method is used to encourage lymphatic drainage, reduce puffiness, reduce muscle tension, tone skin, increase circulation, soften fine lines and wrinkles and increase absorption of facial skin care.
